(19) United States
(12) Patent Application Publication
(10) Pub. No.: US 2020/0337663 A1
(43) Pub. Date: Oct. 29, 2020

(54) MEDICAL IMAGING SYSTEM AND METHOD FOR OPERATING SAME, STORAGE MEDIUM AND PROCESSOR

(71) Applicant: Siemens Healthcare GmbH, Erlangen (DE)

(72) Inventors: Qiong Zhang, Shenzhen (CN); Liu En Yang, Shenzhen (CN); Yuan Yuan Kang, Shenzhen (CN)

(73) Assignee: Siemens Healthcare GmbH, Erlangen (DE)

(21) Appl. No.: 16/858,213

(22) Filed: Apr. 24, 2020

(30) Foreign Application Priority Data
Apr. 24, 2019 (CN) 201910335047.6

(51) Int. Cl. A61B 6/00; A61B 5/055; A61B 6/03; A61B 5/00

(52) U.S. Cl. CPC A61B 6/504; A61B 5/055; A61B 6/032; A61B 5/0037; A61B 5/725; A61B 6/541; A61B 6/5205; A61B 5/489; A61B 6/469

(57) ABSTRACT

Techniques are disclosed for operating a medical imaging system to obtain frames of initial scanned images of a cross section of a scanned site of interest at different times in a first cycle; compare the frames to identify a plurality of blood vessel locations in the frames; calculate, according to the signal strengths of the frames at the plurality of blood vessel locations, a trigger time for triggering the scanning of the medical imaging system in a second cycle; combining the plurality of frames to determine a scanning range for operating the medical imaging system to scan the site of interest during the second cycle; and generating, according to the trigger time and the scanning range, a blood vessel imaged image of the site of interest during the second cycle.

### CROSS-REFERENCE TO RELATED APPLICATIONS

The present application claims the benefit of the filing date of China patent application no. 201910335047.6, filed on Apr. 24, 2019, the contents of which are incorporated herein by reference in their entirety.

### TECHNICAL FIELD

The present disclosure relates to a method for operating a medical imaging system to perform blood vessel imaging scans.

### BACKGROUND

Medical imaging is a technique and process for obtaining an image of an internal tissue in the human body or a part of the human body in a non-invasive manner, and has become an important medical diagnosis technology widely used for all parts of the human body. In the field of medical imaging technology, apparatuses using X-ray imaging technology are referred to as X-ray imaging apparatuses. With the development of computer technology, ordinary X-ray imaging apparatuses can be combined with electronic computers, such that X-ray information is converted from analog to digital information, thereby obtaining digital images. This imaging technology is called digital X-ray imaging technology.

In addition, a medical imaging technology called angiography can also be used to observe the distribution of blood vessels, such as arteries, veins, or atria. Angiography can be used specifically for the diagnosis of vascular disease, and sometimes to determine the structure of a blood vessel before surgery. Angiographic images may be static images, for example, used to examine specific areas; or dynamic images, for example, thirty images are dynamically presented per second, from which the speed of blood flowing through the blood vessels can also be observed. Angiography can photograph the developmental process of a blood vessel cavity by means of television photography or video. The sequence of blood flow and the status of blood vessel filling can be seen from the results of development, and the physiological and anatomical changes of the blood vessel can be learned accordingly. A medical examination method combining angiography and computer technology is thus formed.

### SUMMARY

The present disclosure provides a medical imaging system and a method for operating a medical imaging system. For example, the medical imaging system may be a system that images a blood vessel by using X-ray, CT, or magnetic resonance imaging technology. The present disclosure further provides a storage medium and a processor related to the operating method.

According to an aspect of the present disclosure, a method for operating a medical imaging system is provided, the method comprising: scanning a site of interest of an examined object to obtain a plurality of frames of initial scanned images of a cross section of the site of interest at different times in a first cycle; comparing the plurality of frames of initial scanned images to identify a plurality of blood vessel locations on the plurality of frames of initial scanned images; calculating, according to the signal strengths of the plurality of frames of initial scanned images at the plurality of blood vessel locations, a trigger time for triggering the scanning of the medical imaging system in a second cycle; combining the plurality of frames of initial scanned images to determine a scanning range for operating the medical imaging system to scan the site of interest in the second cycle; and generating, according to the trigger time and the scanning range, a blood vessel imaged image of the site of interest in the second cycle.

The method according to the present disclosure simplifies the work flow of blood vessel imaging, and can determine parameters for formal scanning in a pre-scanning phase by means of fully automated calculation, so that the work flow becomes simple and automatic, and manual intervention is not required. Moreover, the examination process using this technology will become time-saving and efficient, and will not cause time-consuming and inefficient problems due to complicated manual operations.

According to an exemplary embodiment of the method for operating a medical imaging system of the present disclosure, obtaining the plurality of frames of initial scanned images of the cross section of the site of interest at different times in the first cycle comprises: obtaining the plurality of frames of initial scanned images at a predetermined frequency in different phases of a cardiac cycle of the examined object. The obtaining of the plurality of frames of initial scanned images is triggered according to different phases of the cardiac cycle of the examined object, for example, by an electrocardiogram, such that the obtained initial scanned images are associated with cardiac pulses of the examined object, and then associated with the flow velocity of blood at the site of interest of the examined object.

According to an exemplary embodiment of the method for operating a medical imaging system of the present disclosure, identifying the plurality of blood vessel locations on the plurality of frames of initial scanned images comprises: using the initial scanned image obtained in the first phase of the cardiac cycle as a reference image; calculating differences between the initial scanned images obtained in the other phases of the cardiac cycle and the reference image to generate a plurality of frames of differential images; and adding the plurality of frames of differential images to one another to identify the plurality of blood vessel locations. Since background noise (generally assumed to be Gaussian noise with an average value of 0) outside organs or tissues on the scanned images of different phases is considered as randomly distributed values, only random noise is generated by means of the subtraction of two scanned images (for example, the subtraction of a scanned image obtained in another phase of the cardiac cycle from the reference image), but the addition of the images to one another will not cause a high expected value. The same applies to static tissues. Even if these static tissues reflect high signal strengths on the scanned images obtained in different phases of the cardiac cycle, the differential images obtained by subtracting the scanned images obtained in the other phases of the cardiac cycle from the reference image have only the results similar to noise. Therefore, all signals related to blood vessels will become very obvious on the differential level image obtained by adding the differential images to one another, and arteries and veins can also be distinguished very easily on the image according to the differences in blood flow velocities of arterial blood vessels and venous blood vessels, because the sum of the differences in arterial signals is much larger than the sum of the differences in venous signals.

According to an exemplary embodiment of the method for operating a medical imaging system of the present disclosure, calculating the trigger time for triggering the scanning of the medical imaging system in the second cycle comprises: determining a plurality of blood vessel regions related to the plurality of blood vessel locations on each frame of initial scanned image; selecting a target scanned image from the plurality of frames of initial scanned images according to the average signal strength in each of the plurality of blood vessel regions; determining a scanning time of the target scanned image in the first cycle; and calculating the trigger time according to the scanning time. The calculation of the trigger time is based on the average signal strength in the blood vessel regions, rather than the signal strengths of points at the blood vessel locations, and this particularly improves the stability of the calculation process of the trigger time, thereby ensuring the reliability of the imaging system in operation.

According to an exemplary embodiment of the method for operating a medical imaging system of the present disclosure, determining the blood vessel regions related to the plurality of blood vessel locations on each frame of initial scanned image comprises: determining, with each of the blood vessel locations as the center and a predetermined number of pixels as the radius, a plurality of circular blood vessel regions related to the plurality of blood vessel locations on each frame of initial scanned image. Through a location point of a blood vessel located on an initial scanned image, a blood vessel range related to the location point can be delineated. The blood vessel range has a circular shape that facilitates distinguishing and subsequent processing in this embodiment.

According to an exemplary embodiment of the method for operating a medical imaging system of the present disclosure, selecting the target scanned image from the plurality of frames of initial scanned images comprises: determining an initial scanned image comprising a blood vessel region having a maximum average signal strength at the same blood vessel location in the plurality of frames of initial scanned images; and determining the determined initial scanned image as the target scanned image. The blood vessel region of the initial scanned image having the maximum average signal strength represents that the blood peak of the heart just arrives at the scanned site when the initial scanned image is generated. At this time, the blood flow at the scanned site is very fast.

According to an exemplary embodiment of the method for operating a medical imaging system of the present disclosure, determining the scanning time of the target scanned image in the first cycle comprises: determining a frame number of the target scanned image among the plurality of frames of initial scanned images; and calculating, according to the frame number and the predetermined frequency, the scanning time of the target scanned image in the first cycle. Therefore, the scanning time of the target scanned image relative to the plurality of frames of initial scanned images can be determined. In other words, the delay time of the target scanned image relative to the reference image is determined, that is, the duration from the triggering of the scanning to generate the first image to the generation of the target image in the first cycle.

According to an exemplary embodiment of the method for operating a medical imaging system of the present disclosure, calculating the trigger time comprises: calculating an average value of the scanning times of the target scanned images selected for the respective blood vessel locations in the first cycle; and determining the average value as the trigger time. Therefore, the trigger time is not determined by the scanning time of the target scanned image for a specific blood vessel location, but by the scanning time corresponding to each of the identified plurality of blood vessel locations, which further improves the stability of the system.

According to an exemplary embodiment of the method for operating a medical imaging system of the present disclosure, determining the scanning range for operating the medical imaging system to scan the site of interest in the second cycle comprises: combining a first plurality of frames of initial scanned images of a cross section at a first position of the site of interest and a second plurality of frames of initial scanned images of a cross section at a second position thereof to generate a first mask image and a second mask image respectively; determining a first center line of the first mask image and a second center line of the second mask image; aligning the first center line and the second center line with a predetermined field of view respectively to align the first mask image and the second mask image with the predetermined field of view respectively; and aligning the predetermined field of view aligned on the first mask image with the predetermined field of view aligned on the second mask image to determine the scanning range. The scanned images obtained from the cross sections at two positions of the site of interest are aligned to locate the site of interest to be formally scanned in a three-dimensional space and determine the scanning range in the three-dimensional space.

According to an exemplary embodiment of the method for operating a medical imaging system of the present disclosure, generating the first mask image and the second mask image comprises: processing the first plurality of frames of initial scanned images and the second plurality of frames of initial scanned images by using a Gaussian filter; synthesizing the processed first plurality of frames of initial scanned images and the processed second plurality of frames of initial scanned images respectively into a first synthesized image and a second synthesized image; and segmenting, with blank regions containing no tissues in the first synthesized image and the second synthesized image as background noise references, the first synthesized image and the second synthesized image to generate the first mask image and the second mask image, respectively. The generation of the mask images facilitates the determination of the center lines of the site of interest in the scanned images, so as to align the cross sections of the site of interest in the images scanned at different positions.

According to an exemplary embodiment of the method for operating a medical imaging system of the present disclosure, the first mask image and the second mask image each comprises two tissue regions arranged about a central axis and containing tissues, wherein determining the first center line of the first mask image and the second center line of the second mask image comprises: calculating, according to the average value of coordinates of the first tissue region and second tissue region of the first mask image, a first center of the first tissue region and a second center of the second tissue region; calculating, according to the average value of coordinates of the third tissue region and fourth tissue region of the second mask image, a third center of the third tissue region and a fourth center of the fourth tissue region; determining the center line of the first mask image according to the connecting line between the first center and the second center and the central axis; and determining the center line of the second mask image according to the connecting line between the third center and the fourth center and the central axis. This makes it easy to determine the center lines of the mask images by means of automated calculation, thereby facilitating the alignment between a plurality of mask images in subsequent steps.

According to an exemplary embodiment of the method for operating a medical imaging system of the present disclosure, generating the blood vessel imaged image of the site of interest comprises: generating a first imaged image of the site of interest in the first phase of the cardiac cycle; generating a second imaged image of the site of interest at the trigger time; and subtracting the first imaged image from the second imaged image to generate the blood vessel imaged image of the site of interest. When two images are subtracted from each other, static tissue signals in the site of interest on the scanned images are canceled, and the blood vessels can be therefore easily identified from the subtracted image.

According to an exemplary embodiment of the method for operating a medical imaging system of the present disclosure, the plurality of frames of initial scanned images are two-dimensional FLASH movie images. In the case of using magnetic resonance technology, a two-dimensional cross-sectional movie of the site of interest is obtained by using a FLASH sequence, which facilitates the identification of blood vessel locations and clinical locations in the formal scanning by processing the movie images in subsequent steps.

According to an exemplary embodiment of the method for operating a medical imaging system of the present disclosure, the blood vessels are arterial blood vessels. With the method according to the present disclosure, in particular, an imaged image of arterial blood vessels, for example, a peripheral arterial blood vessel imaged image, can be obtained.

According to another aspect of the present disclosure, the present disclosure also provides a medical imaging system, comprising: a scanning device (e.g. an imager or scanner) configured to scan a site of interest of an examined object to obtain a plurality of frames of initial scanned images of a cross section of the site of interest at different times in a first cycle; a blood vessel location identification device configured to compare the plurality of frames of initial scanned images to identify a plurality of blood vessel locations on the plurality of frames of initial scanned images; a trigger time calculation device configured to calculate, according to the signal strengths of the plurality of frames of initial scanned images at the plurality of blood vessel locations, a trigger time for triggering the scanning of the medical imaging system in a second cycle; a scanning range determination device configured to combine the plurality of frames of initial scanned images to determine a scanning range for operating the medical imaging system to scan the site of interest in the second cycle; and an image generation device configured to generate, according to the trigger time and the scanning range, a blood vessel imaged image of the site of interest in the second cycle. The medical imaging system according to the present disclosure can particularly perform a simplified work flow of blood vessel imaging, and particularly determine parameters for formal scanning in a pre-scanning phase by means of fully automated calculation, so that the work flow becomes simple and automatic, and manual intervention is not required.

According to an exemplary embodiment of the medical imaging system of the present disclosure, the scanning device is further configured to: obtain the plurality of frames of initial scanned images at a predetermined frequency in different phases of a cardiac cycle of the examined object. The obtaining of the plurality of frames of initial scanned images is triggered according to different phases of the cardiac cycle of the examined object, for example, by an electrocardiogram.

According to an exemplary embodiment of the medical imaging system of the present disclosure, the blood vessel location identification device is further configured to: use the initial scanned image obtained in the first phase of the cardiac cycle as a reference image; calculate differences between the initial scanned images obtained in the other phases of the cardiac cycle and the reference image to generate a plurality of frames of differential images; and adding the plurality of frames of differential images to one another to identify the plurality of blood vessel locations. All signals related to blood vessels will become very obvious on the differential level image obtained by adding the differential images to one another, and arteries and veins can also be distinguished very easily on the image according to the differences in blood flow velocities of arterial blood vessels and venous blood vessels.

According to an exemplary embodiment of the medical imaging system of the present disclosure, the trigger time calculation device is further configured to: determine a plurality of blood vessel regions related to the plurality of blood vessel locations on each frame of initial scanned image; select a target scanned image from the plurality of frames of initial scanned images according to the average signal strength in each of the plurality of blood vessel regions; determine a scanning time of the target scanned image in the first cycle; and calculate the trigger time according to the scanning time. The trigger time is calculated based on the average signal strength in the blood vessel regions, rather than the signal strengths of points on the blood vessel locations, which ensures the reliability of the imaging system in operation.

According to an exemplary embodiment of the medical imaging system of the present disclosure, the trigger time calculation device is further configured to: determine, with each of the blood vessel locations as the center and a predetermined number of pixels as the radius, a plurality of circular blood vessel regions related to the plurality of blood vessel locations on each frame of initial scanned image. Through a location point of a blood vessel located on an initial scanned image, a blood vessel range related to the location point can be delineated. The blood vessel range has a circular shape that facilitates distinguishing and subsequent processing in this embodiment.

According to an exemplary embodiment of the medical imaging system of the present disclosure, the trigger time calculation device is further configured to: determine an initial scanned image comprising a blood vessel region having a maximum average signal strength at the same blood vessel location in the plurality of frames of initial scanned images; and determine the determined initial scanned image as the target scanned image. The blood vessel region of the initial scanned image having the maximum average signal strength represents that the blood peak of the heart just arrives at the scanned site when the initial scanned image is generated.

According to an exemplary embodiment of the medical imaging system of the present disclosure, the trigger time calculation device is further configured to: determine a frame number of the target scanned image among the plurality of frames of initial scanned images; and calculate, according to the frame number and the predetermined frequency, the scanning time of the target scanned image in the first cycle. Therefore, the delay time of the target scanned image relative to the reference image can be determined, that is, the duration from the triggering of the scanning to generate the first image to the generation of the target image in the first cycle.

According to an exemplary embodiment of the medical imaging system of the present disclosure, the trigger time calculation device is further configured to: calculate an average value of the scanning times of the target scanned images selected for the respective blood vessel locations in the first cycle; and determine the average value as the trigger time. The trigger time is determined by the scanning time corresponding to each of the identified plurality of blood vessel locations, which further improves the stability of the system.

According to an exemplary embodiment of the medical imaging system of the present disclosure, the scanning range determination device is further configured to: combine a first plurality of frames of initial scanned images of a cross section at a first position of the site of interest and a second plurality of frames of initial scanned images of a cross section at a second position thereof to generate a first mask image and a second mask image respectively; determine a first center line of the first mask image and a second center line of the second mask image; align the first center line and the second center line with a predetermined field of view respectively to align the first mask image and the second mask image with the predetermined field of view respectively; and align the predetermined field of view aligned on the first mask image with the predetermined field of view aligned on the second mask image to determine the scanning range. The scanned images obtained from the cross sections at two positions of the site of interest are aligned to locate the site of interest to be formally scanned in a three-dimensional space and determine the scanning range in the three-dimensional space.

According to an exemplary embodiment of the medical imaging system of the present disclosure, the scanning range determination device is further configured to: process the first plurality of frames of initial scanned images and the second plurality of frames of initial scanned images by using a Gaussian filter; synthesize the processed first plurality of frames of initial scanned images and the processed second plurality of frames of initial scanned images respectively into a first synthesized image and a second synthesized image; and segment, with blank regions containing no tissues in the first synthesized image and the second synthesized image as background noise references, the first synthesized image and the second synthesized image to generate the first mask image and the second mask image respectively. The generation of the mask images facilitates the determination of the center lines of the site of interest in the scanned images, so as to align the cross sections of the site of interest in the images scanned at different positions.

According to an exemplary embodiment of the medical imaging system of the present disclosure, the first mask image and the second mask image each comprises two tissue regions arranged about a central axis and containing tissues, wherein the scanning range calculation device is further configured to: calculate, according to the average value of coordinates of the first tissue region and second tissue region of the first mask image, a first center of the first tissue region and a second center of the second tissue region; calculate, according to the average value of coordinates of the third tissue region and fourth tissue region of the second mask image, a third center of the third tissue region and a fourth center of the fourth tissue region; determine the center line of the first mask image according to the connecting line between the first center and the second center and the central axis; and determine the center line of the second mask image according to the connecting line between the third center and the fourth center and the central axis. This makes it easy to determine the center lines of the mask images by means of automated calculation, thereby facilitating the alignment between a plurality of mask images in subsequent steps.

According to an exemplary embodiment of the medical imaging system of the present disclosure, the image generation device is further configured to: generate a first imaged image of the site of interest in the first phase of the cardiac cycle; generate a second imaged image of the site of interest at the trigger time; and subtract the first imaged image from the second imaged image to generate the blood vessel imaged image of the site of interest. When two images are subtracted from each other, static tissue signals in the site of interest on the scanned images are canceled, and the blood vessels with dynamic signals can be therefore easily identified from the subtracted image.

According to another aspect of the present disclosure, a storage medium (e.g. a non-transitory computer-readable medium) is provided, comprising a stored program or executable and/or readable instructions stored thereon, wherein the program, when running or otherwise executed by one or more processors, controls an apparatus where the storage medium is located to perform the method described above.

According to another aspect of the present disclosure, one or more processors are provided that are configured to run a program or otherwise execute the instructions stored in the computer-readable medium, wherein the method described above is performed when the program runs and/or when the instructions are executed in this manner.

### BRIEF DESCRIPTION OF THE DRAWINGS/FIGURES

The accompanying drawings constitute a part of the description to facilitate further understanding of the present disclosure. These drawings illustrate the embodiments of the present disclosure and, together with the description, explain the principles of the present disclosure. In the drawings, the same parts are denoted by the same signs. In the figures:

FIG. 1 shows a schematic flowchart of the operation of a medical imaging system according to an embodiment of the present disclosure.

FIG. 2 shows a schematic flowchart of a method for calculating a trigger time for triggering the scanning of the medical imaging system according to an exemplary embodiment of the present disclosure.

FIG. 3 shows a schematic flowchart of a method for determining a scanning range of a site of interest according to an exemplary embodiment of the present disclosure.

FIGS. 4A-4C show schematic diagrams of imaged images obtained by implementing the exemplary flow described in FIG. 2.

FIG. 5 shows a schematic diagram of imaged images obtained by implementing the exemplary flow described in FIG. 3.

FIG. 6 shows a schematic structural diagram of a medical imaging system according to an embodiment of the present disclosure.

### DESCRIPTION OF REFERENCE SIGNS

100: Medical imaging system
101: Scanning device
103: Blood vessel location identification device
105: Trigger time calculation device
107: Scanning range determination device
109: Image generation device.

### DETAILED DESCRIPTION

In order for a person skilled in the art to better understand the solutions of the present disclosure, the technical solutions in the embodiments of the present disclosure will be clearly and completely described below with reference to the accompanying drawings for the embodiments of the present disclosure. Apparently, the described embodiments are merely some of, rather than all of, the embodiments of the present disclosure. All other solutions obtained by those of ordinary skill in the art based on the embodiments of the present disclosure without involving any creative effort shall fall within the scope of protection of the present disclosure.

It should be noted that the terms “comprise” and “have” and any variations thereof in the description and the claims of the present disclosure are intended to cover a non-exclusive inclusion, for example, a product or apparatus including a series of units is not necessarily limited to those units listed, but may comprise other units not listed or inherent to such a product or apparatus.

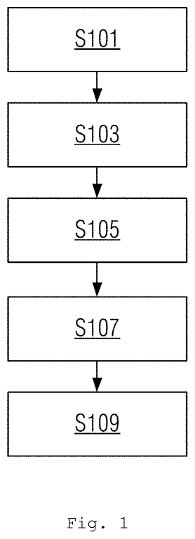

FIG. 1 shows a schematic flowchart of the operation of a medical imaging system according to an embodiment of the present disclosure. A method for operating a medical imaging system according to an embodiment of the present disclosure comprises:

Step S101, scanning a site of interest of an examined object to obtain a plurality of frames of initial scanned images of a cross section of the site of interest at different times in a first cycle. For example, in this embodiment, in the case of using magnetic resonance (MR) technology, a two-dimensional cross-sectional movie of the site of interest of the examined object is obtained using a FLASH pulse sequence.

Step S103, comparing the plurality of frames of initial scanned images to identify a plurality of blood vessel locations on the plurality of frames of initial scanned images. Location information about a plurality of blood vessels on the cross section of the site of interest can be obtained from the two-dimensional cross-sectional movie of the site of interest, and in particular, location information about a plurality of arterial blood vessels on the cross section can be obtained.

Step S105, calculating, according to the signal strengths of the plurality of frames of initial scanned images at the plurality of blood vessel locations, a trigger time for triggering the scanning of the medical imaging system in a second cycle. According to the pre-scanned images obtained in the first cycle, the trigger time for triggering the scanning in the second cycle, that is, the subsequent cycle can be determined, wherein the trigger time is particularly determined depending on the signal strengths at the blood vessel locations on the scanned images.

Step S107, combining the plurality of frames of initial scanned images to determine a scanning range for operating the medical imaging system to scan the site of interest in the second cycle. The scanning range used in the formal clinical examination of the subsequent cycle is determined according to the scanned images obtained in the pre-scanning phase of the first cycle.

Step S109, generating, according to the trigger time and the scanning range, a blood vessel imaged image of the site of interest in the second cycle. The blood vessel imaged image is an image particularly generated at the time of formal clinical examination, and this image shows three-dimensional imaging of arterial blood vessels at the site of interest. In order to generate the blood vessel imaged image of the site of interest, a first imaged image of the site of interest in a first phase of a cardiac cycle needs to be generated first, and then a second imaged image of the site of interest at the trigger time is generated. Accordingly, the first imaged image is subtracted from the second imaged image to generate the blood vessel imaged image of the site of interest.

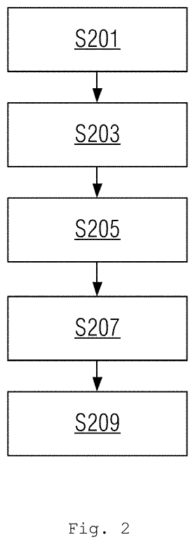

FIG. 2 shows a schematic flowchart of a method for calculating a trigger time for triggering the scanning of the medical imaging system according to an exemplary embodiment of the present disclosure. A method for calculating a trigger time according to an exemplary embodiment of the present disclosure comprises:

Step S201, obtaining, at a predetermined frequency in different phases of a cardiac cycle of an examined object, a plurality of frames of initial scanned images of a cross section of a site of interest of the examined object at different times in a first cycle. For example, a scanning of the cross section of the site of interest is triggered according to an electrocardiogram (ECG) of the examined object.

Step S203, using the initial scanned image obtained in the first phase of the cardiac cycle as a reference image, and calculating differences between the initial scanned images obtained in the other phases of the cardiac cycle and the reference image to generate a plurality of frames of differential images. Subsequently, the plurality of frames of differential images are added to one another to identify a plurality of blood vessel locations. Only signals of changes in blood vessels are on the differential images, whereas static tissues behave like noise on the differential images, and can be approximately regarded as noise with an average value of 0. In the first phase of the cardiac cycle, a trigger signal of ECG has just been received, and a scanning of the cross section of the site of interest is triggered according to the signal. Thereafter, scanned images of the cross section of the site of interest in the other phases of the cardiac cycle are also obtained for comparison with the reference image to obtain differences between the images. The obtained differential images are added to one another to obtain a differential level image on which the locations of arterial blood vessels can be clearly detected or observed.

Step S205, determining a plurality of blood vessel regions related to the plurality of blood vessel locations on each frame of initial scanned image, selecting a target scanned image from the plurality of frames of initial scanned images according to the average signal strength in each of the plurality of blood vessel regions, and determining a scanning time of the target scanned image in the first cycle. Accordingly, the trigger time is calculated according to the scanning time. In order to determine a plurality of blood vessel regions related to the plurality of blood vessel locations on each frame of initial scanned image, each blood vessel location is used as the center and a predetermined number of pixels are used as the radius to determine a plurality of circular blood vessel regions related to the plurality of blood vessel locations on each frame of initial scanned image. In order to select a target scanned image, an initial scanned image comprising a blood vessel region having a maximum average signal strength at the same blood vessel location in the plurality of frames of initial scanned images is determined first, and then the determined initial scanned image is determined as the target scanned image.

Step S207, determining a frame number of the target scanned image among the plurality of frames of initial scanned images; and calculating, according to the frame number and the predetermined frequency, the scanning time of the target scanned image in the first cycle. After the appropriate target scanned image is selected, in order to determine a delay of the target scanned image relative to the reference image obtained in the first cycle, the serial number of the target scanned image among the plurality of frames of initial scanned images obtained in the first cycle needs to be determined, that is, the frame number of the target scanned image among the plurality of frames of initial scanned images needs to be determined.

Step S209, calculating an average value of the scanning times of the target scanned images selected for the respective blood vessel locations in the first cycle; and determining the average value as the trigger time. A target scanned image related to each blood vessel location is determined. For example, three arterial blood vessel locations can be determined according to the two-dimensional scanned images of the cross section. For the three arterial blood vessel locations, three appropriate target scanned images are determined respectively, respective scanning times of the three target scanned images are then determined, and finally, an average value of the three scanning times is calculated.

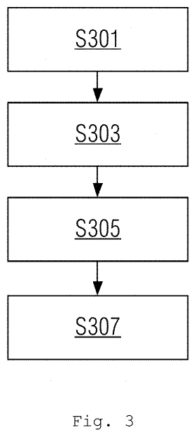

FIG. 3 shows a schematic flowchart of a method for determining a scanning range of a site of interest according to an exemplary embodiment of the present disclosure. A method for determining a scanning range according to an exemplary embodiment of the present disclosure comprises:

Step S301, obtaining, at a predetermined frequency in different phases of a cardiac cycle of an examined object, a plurality of frames of initial scanned images of cross sections, at different positions, of a site of interest of the examined object at different times in a first cycle. In other words, in this step, a first plurality of frames of initial scanned images of a cross section at a first position of the site of interest and a second plurality of frames of initial scanned images of a cross section at a second position thereof are obtained.

Step S303, combining the first plurality of frames of initial scanned images of the cross section at the first position of the site of interest and the second plurality of frames of initial scanned images of the cross section at the second position thereof to generate a first mask image and a second mask image, respectively. For example, the first plurality of frames of initial scanned images and the second plurality of frames of initial scanned images are first processed using a Gaussian filter, and the processed first plurality of frames of initial scanned images and the processed second plurality of frames of initial scanned images are then respectively synthesized into a first synthesized image and a second synthesized image. Finally, with blank regions containing no tissues in the first synthesized image and the second synthesized image as background noise references, the first synthesized image and the second synthesized image are segmented so as to generate a first mask image and a second mask image, respectively.

Step S305, determining a first center line of the first mask image and a second center line of the second mask image. For example, the first mask image and the second mask image each comprises two tissue regions arranged about a central axis and containing tissues. A first center of the first tissue region and a second center of the second tissue region are calculated according to the average value of coordinates of the first tissue region and second tissue region of the first mask image, and a third center of the third tissue region and a fourth center of the fourth tissue region are calculated according to the average value of coordinates of the third tissue region and fourth tissue region of the second mask image. Subsequently, the center line of the first mask image is determined according to the connecting line between the first center and the second center and the central axis, and the center line of the second mask image is determined according to the connecting line between the third center and the fourth center and the central axis.

Step S307, aligning the first center line and the second center line with a predetermined field of view respectively to align the first mask image and the second mask image with the predetermined field of view respectively, and aligning the predetermined field of view aligned on the first mask image with the predetermined field of view aligned on the second mask image to determine the scanning range.

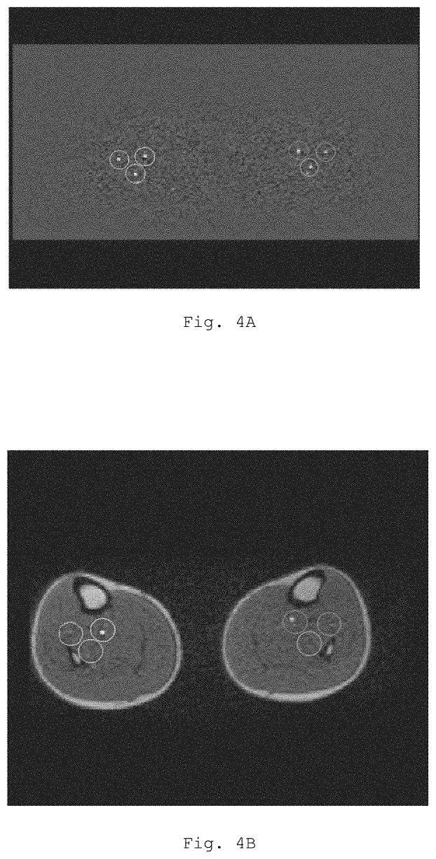

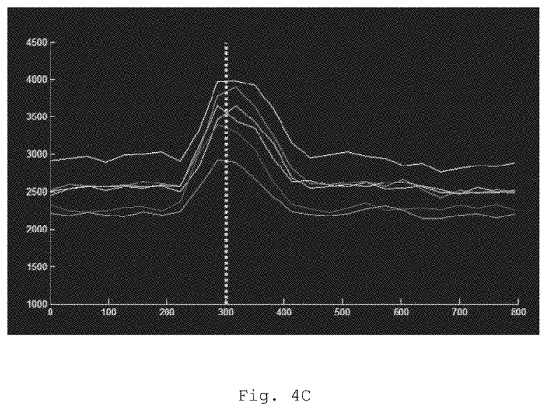

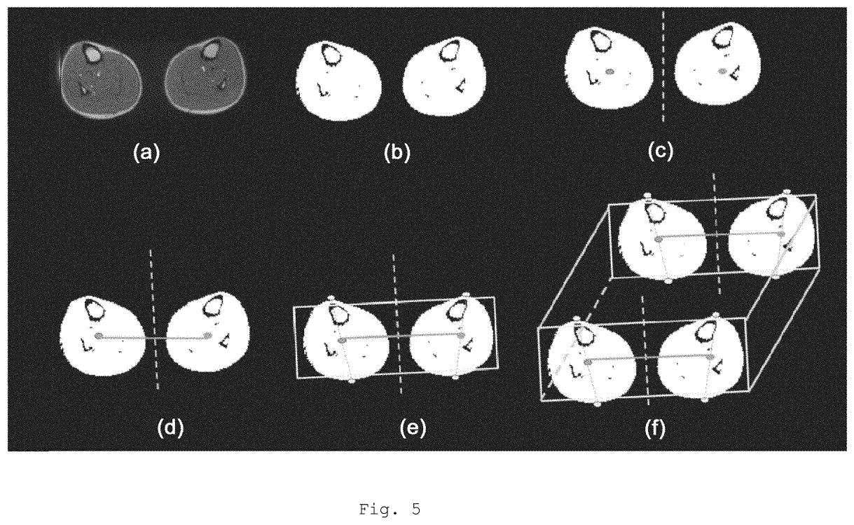

FIGS. 4A-4C show schematic diagrams of imaged images obtained by implementing the exemplary flow described in FIG. 2. FIG. 4A shows a differential level image obtained after a plurality of frames of initial scanned images regarding two-dimensional movie images of a cross section of a leg are processed, where a plurality of bright spots representing arterial blood vessel locations can be clearly detected or observed on the image. Moreover, in the left and right regions of FIG. 4A, circles are drawn with the arterial blood vessel locations represented by the bright spots as the centers and 4 or 5 pixels as the radius, and the circles represent the cross sections of the arterial blood vessels. In FIG. 4B, the determined cross sections of the arterial blood vessels are mapped onto original initial scanned images to determine average values of signal strengths within these cross sections. In FIG. 4C, for each arterial blood vessel location, a signal change curve is determined according to the average value of signal strengths with the cross section related to this location and the corresponding scanning time of each frame of scanned image. As shown in the signal curve diagram of FIG. 4C, the abscissa axis represents the scanning times of a plurality of frames of scanned image, that is, the delay time relative to the first scanning in the first cycle of the pre-scanning phase, and the ordinate axis represents the signal strength for one of blood vessel locations at the corresponding scanning time. Subsequently, the scanned image corresponding to the maximum value of each change curve is determined as a target scanned image corresponding to the current arterial blood vessel location. Further, a trigger time for triggering a scanning in a second cycle is determined according to the average value of corresponding trigger times of the target scanned images selected for all the blood vessel locations.

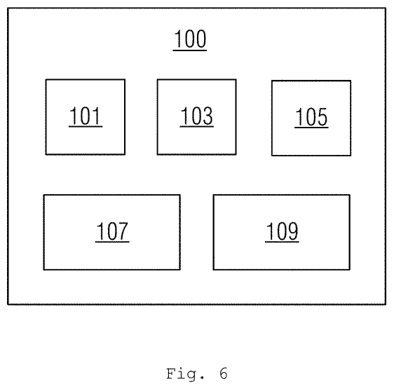

FIG. 5 shows a schematic diagram of imaged images obtained by implementing the exemplary flow described in FIG. 3. In FIG. 5(a), a plurality of frames of scanned images as two-dimensional FLASH movie images of the cross section at the first position of the leg obtained in the pre-scanning phase are processed using a Gaussian filter. The filtered movie images are added to one another to construct a combined image, and a mask image corresponding to the cross section at the first position of the leg is then calculated using an upper left or lower right region of the image as noise, as shown in FIG. 5(b). In FIG. 5(c), for example, since the legs of the examined object are physically separated (for example, separated by a PA coil), the images of the legs can be easily separated in the mask images. In this figure, the mask images of the left leg and the right leg are separated by a dotted line. In this way, a center corresponding to the mask image of the left leg and a center corresponding to the mask image of the right leg are determined by calculating an average value of coordinates of the left and right leg images in the mask images. In (d)-(e) of FIG. 5, the centers of the mask images of the legs are connected, where the connecting line between the centers is perpendicular to the dotted line in (d), then a center line of the mask image constituted by the dotted line and the connecting line between the centers is determined, and the predetermined field of view represented by a rectangular box is aligned with the determined center line. By repeating (a)-(e), two-dimensional FLASH movie images of the cross section at the second position of the leg obtained in the pre-scanning phase are processed in a similar way. Subsequently, in (f) of FIG. 5, the aligned mask images corresponding to the two positions of the legs are aligned, thereby determining a rectangular parallelepiped scanning range in the three-dimensional space as shown in the figure.

FIG. 6 shows a schematic structural diagram of a medical imaging system according to an embodiment of the present disclosure. The medical imaging system 100 as further discussed herein may include various components, which are referred to herein as “devices.” These devices may be implemented as any suitable number and type of computer processors, and may perform their respective functions via hardware components, software components, or combinations thereof. Therefore, each of the devices as shown and described with reference to FIG. 6 may alternatively be referred to as “units,” “circuitry,” or “modules.”

In the embodiment of the present disclosure, as shown in FIG. 6, the medical imaging system 100 comprises: a scanning device 101 configured to scan a site of interest of an examined object to obtain a plurality of frames of initial scanned images of a cross section of the site of interest at different times in a first cycle; a blood vessel location identification device 103 configured to compare the plurality of frames of initial scanned images to identify a plurality of blood vessel locations on the plurality of frames of initial scanned images; a trigger time calculation device 105 configured to calculate, according to the signal strengths of the plurality of frames of initial scanned images at the plurality of blood vessel locations (e.g., pixels or other points at blood vessel locations in the images), a trigger time for triggering the scanning of the medical imaging system in a second cycle; a scanning range determination device 107 configured to combine the plurality of frames of initial scanned images to determine a scanning range for operating the medical imaging system to scan the site of interest in the second cycle; and an image generation device 109 configured to generate, according to the trigger time and the scanning range, a blood vessel imaged image of the site of interest in the second cycle. The term “signal strength” as used in this context and elsewhere throughout the present disclosure may be identified, for instance, as an absolute value that is represented in the image and is associated with a value at particular respective points (e.g., pixels or other points at blood vessel locations in the images). The signal strength and the point or pixel are essentially the same, but there may be some adjustments when converting the signal strength to the value of the point in the image, such as rounding due to the requirements of accuracy, or normalization or dynamic range adjustment due to digitization. Of course, this adjustment will not change the regularity of the signal itself.

The medical imaging system 100 is shown herein by way of example and not limitation, and may include additional, alternate, or fewer components than those shown in FIG. 6. For instance, although not shown in FIG. 6 for purposes of brevity, the medical imaging system 100 may include the storage medium (e.g. a non-transitory computer-readable medium) as discussed herein, which may include a stored program or executable and/or readable instructions stored thereon, wherein the program, when running or otherwise executed by one or more processors and/or the devices of the medical imaging system 100 as shown in FIG. 6, performs the method described above. Thus, the medical imaging system 100 and the internal devices thereof described in FIG. 6 may perform the method for operating the medical imaging system shown in FIGS. 1, 2 and 3, the details of which need not be repeated here.

In the embodiments provided by the present disclosure, it should be understood that the disclosed technical content may be implemented in other ways. The device embodiments described above are merely schematic. For example, the division of the units or modules is only a logical function division, and in actual implementations, there may be other division methods. For example, a plurality of units or modules or components may be combined or integrated into another system, or some features may be omitted or not implemented. In addition, the mutual coupling or direct coupling shown or discussed may be an indirect coupling through some interfaces, modules or units, and may be electrical or in other forms.

The foregoing description is merely illustrative of the preferred examples of the disclosure and is not intended to limit the disclosure, and various changes and modifications may be made by a person skilled in the art. Any modifications, equivalent substitutions, improvements, etc. made within the spirit and principles of the present disclosure shall fall into the scope of protection of the present disclosure.

What is claimed is:

1. A method for operating a medical imaging system, comprising:
scanning a site of interest of an examined object to obtain a plurality of frames of initial scanned images of a cross section of the site of interest at different times during a first cycle;
comparing each of the plurality of frames of the initial scanned images to one another to identify a plurality of blood vessel locations in the plurality of frames of the initial scanned images;
calculating a trigger time for triggering the scanning of the medical imaging system in a second cycle using signal strengths associated with each of the plurality of blood vessel locations in the plurality of frames of the initial scanned images;
combining each of the plurality of frames of the initial scanned images to determine a scanning range for operating the medical imaging system to scan the site of interest during the second cycle; and
generating a blood vessel imaged image of the site of interest during the second cycle using the trigger time and the scanning range.

2. The method as claimed in claim 1, wherein obtaining the plurality of frames of the initial scanned images of the cross section of the site of interest at different times during the first cycle comprises:
obtaining the plurality of frames of the initial scanned images at a predetermined frequency in different phases of a cardiac cycle of the examined object.

3. The method as claimed in claim 2, wherein identifying the plurality of blood vessel locations in each of the plurality of frames of the initial scanned images comprises:
using a first one of the initial scanned images obtained during a first phase of the cardiac cycle as a reference image;
calculating differences between (i) the initial scanned images obtained in other phases of the cardiac cycle, and (ii) the reference image, to generate a plurality of frames of differential images; and
adding the plurality of frames of the differential images to one another to identify the plurality of blood vessel locations.

4. The method as claimed in claim 2, wherein calculating the trigger time comprises:
determining a plurality of blood vessel regions related to the plurality of blood vessel locations on each frame of the initial scanned images;
selecting a target scanned image from the plurality of frames of the initial scanned images having an average signal strength of a signal strength of each of the plurality of blood vessel regions;
determining a scanning time of the target scanned image during the first cycle; and
calculating the trigger time as the scanning time.

5. The method as claimed in claim 4, wherein determining the blood vessel regions related to the plurality of blood vessel locations in each frame of the initial scanned images comprises:
determining, with each of the blood vessel locations as the center and a predetermined number of pixels as the radius, a plurality of circular blood vessel regions related to the plurality of blood vessel locations in each frame of the initial scanned images.

6. The method as claimed in claim 4, wherein selecting the target scanned image from the plurality of frames of initial scanned images comprises:
determining an initial scanned image from among the initial scanned images comprising a blood vessel region having a maximum average signal strength at the same blood vessel location in the plurality of frames of the initial scanned images; and
determining the initial scanned image as the target scanned image.

7. The method as claimed in claim 4, wherein determining the scanning time of the target scanned image during the first cycle comprises:
determining a frame number of the target scanned image among the plurality of frames of the initial scanned images; and
calculating, using the frame number and the predetermined frequency, the scanning time of the target scanned image during the first cycle.

8. The method as claimed in claim 4, wherein calculating the trigger time comprises:
calculating an average value of the scanning times of the target scanned images selected for the respective blood vessel locations during the first cycle; and
determining the average value as the trigger time.

9. The method as claimed in claim 1, wherein determining the scanning range for operating the medical imaging system to scan the site of interest during the second cycle comprises:
combining a first plurality of frames of initial scanned images of a cross section at a first position of the site of interest and a second plurality of frames of initial scanned images of a cross section at a second position thereof to generate a first mask image and a second mask image, respectively;
determining a first center line of the first mask image and a second center line of the second mask image;
aligning the first center line and the second center line, respectively, with a predetermined field of view to align the first mask image and the second mask image, respectively, with the predetermined field of view; and
aligning the predetermined field of view aligned on the first mask image with the predetermined field of view aligned on the second mask image to determine the scanning range.

10. The method as claimed in claim 9, wherein generating the first mask image and the second mask image comprises:
processing the first plurality of frames of the initial scanned images and the second plurality of frames of the initial scanned images using a Gaussian filter;
synthesizing the processed first plurality of frames of the initial scanned images and the processed second plurality of frames of the initial scanned images, respectively, into a first synthesized image and a second synthesized image; and
segmenting, with blank regions containing no tissues during the first synthesized image and the second synthesized image as background noise references, the first synthesized image and the second synthesized image, to generate the first mask image and the second mask image, respectively.

11. The method as claimed in claim 10, wherein the first mask image and the second mask image each comprises two tissue regions arranged about a central axis and containing tissues, and wherein determining the first center line of the first mask image and determining the second center line of the second mask image comprises:
calculating, using an average value of coordinates of the first tissue region and the second tissue region of the first mask image, a first center of the first tissue region, and a second center of the second tissue region;
calculating, using an average value of coordinates of the third tissue region and the fourth tissue region of the second mask image, a third center of the third tissue region, and a fourth center of the fourth tissue region;
determining the center line of the first mask image using a connecting line between the first center and the second center, and the central axis; and
determining the center line of the second mask image using a connecting line between the third center and the fourth center, and the central axis.

12. The method as claimed in claim 2, wherein generating the blood vessel imaged image of the site of interest comprises:
generating a first imaged image of the site of interest during a first phase of the cardiac cycle;
generating a second imaged image of the site of interest at the trigger time; and
subtracting the first imaged image from the second imaged image to generate the blood vessel imaged image of the site of interest.

13. The method as claimed in claim 1, wherein each of the plurality of frames of the initial scanned images are two-dimensional FLASH movie images.

14. The method as claimed in claim 1, wherein the blood vessels are arterial blood vessels.

15. A medical imaging system, comprising:
a scanner configured to scan a site of interest of an examined object to obtain a plurality of frames of initial scanned images of a cross section of the site of interest at different times during a first cycle;
blood vessel location identification circuitry configured to compare each of the plurality of frames of the initial scanned images to one another to identify a plurality of blood vessel locations in the plurality of frames of the initial scanned images;
trigger time calculation circuitry configured to calculate a trigger time for triggering the scanning of the medical imaging system in a second cycle using signal strengths associated with each of the plurality of blood vessel locations in the plurality of frames of the initial scanned images;
scanning range determination circuitry configured to combine the plurality of frames of the initial scanned images to determine a scanning range for operating the medical imaging system to scan the site of interest during the second cycle; and
image generation circuitry configured to generate a blood vessel imaged image of the site of interest during the second cycle using the trigger time and the scanning range.

16. The medical imaging system as claimed in claim 15, wherein the scanner is further configured to obtain the plurality of frames of the initial scanned images at a predetermined frequency in different phases of a cardiac cycle of the examined object.

17. The medical imaging system as claimed in claim 16, wherein the blood vessel location identification circuitry is further configured to:
use a first one of the initial scanned images obtained during a first phase of the cardiac cycle as a reference image;
calculate differences between (i) the initial scanned images obtained in the other phases of the cardiac cycle, and (ii) the reference image, to generate a plurality of frames of differential images; and
adding the plurality of frames the differential images to one another to identify the plurality of blood vessel locations.

18. The medical imaging system as claimed in claim 16, wherein the trigger time calculation circuitry is further configured to:
determine a plurality of blood vessel regions related to the plurality of blood vessel locations on each frame of the initial scanned images;
select a target scanned image from the plurality of frames of the initial scanned images having an average signal strength of a signal strength of each of the plurality of blood vessel regions;
determine a scanning time of the target scanned image during the first cycle; and
calculate the trigger time as the scanning time.

19. The medical imaging system as claimed in claim 18, wherein the trigger time calculation circuitry is further configured to determine, with each of the blood vessel locations as the center and a predetermined number of pixels as the radius, a plurality of circular blood vessel regions related to the plurality of blood vessel locations in each frame the initial scanned images.

20. The medical imaging system as claimed in claim 18, wherein the trigger time calculation circuitry is further configured to:
determine a first scanned image from among the initial scanned images comprising a blood vessel region having a maximum average signal strength at the same blood vessel location in the plurality of frames of the initial scanned images; and
determine the determined initial scanned image as the target scanned image.

21. The medical imaging system as claimed in claim 18, wherein the trigger time calculation circuitry is further configured to:
determine a frame number of the target scanned image among the plurality of frames of the initial scanned images; and
calculate, using the frame number and the predetermined frequency, the scanning time of the target scanned image during the first cycle.

22. The medical imaging system as claimed in claim 18, wherein the trigger time calculation circuitry is further configured to:
calculate an average value of the scanning times of the target scanned images selected for the respective blood vessel locations during the first cycle; and
determine the average value as the trigger time.

23. The medical imaging system as claimed in claim 15, wherein the scanning range determination device is further configured to:
combine a first plurality of frames of initial scanned images of a cross section at a first position of the site of interest and a second plurality of frames of initial scanned images of a cross section at a second position thereof to generate a first mask image and a second mask image respectively;
determine a first center line of the first mask image and a second center line of the second mask image;
align the first center line and the second center line, respectively, with a predetermined field of view to align the first mask image and the second mask image, respectively, with the predetermined field of view; and
align the predetermined field of view aligned on the first mask image with the predetermined field of view aligned on the second mask image to determine the scanning range.

24. The medical imaging system as claimed in claim 23, wherein the scanning range determination circuitry is further configured to:
process the first plurality of frames of the initial scanned images and the second plurality of frames of the initial scanned images using a Gaussian filter;
synthesize the processed first plurality of frames of the initial scanned images and the processed second plurality of frames of the initial scanned images, respectively, into a first synthesized image and a second synthesized image; and
segment, with blank regions containing no tissues in the first synthesized image and the second synthesized image as background noise references, the first synthesized image and the second synthesized image to generate the first mask image and the second mask image, respectively.

25. The medical imaging system as claimed in claim 24, wherein the first mask image and the second mask image each comprises two tissue regions arranged about a central axis and containing tissues, and
wherein the scanning range calculation circuitry is further configured to:
calculate, using an average value of coordinates of the first tissue region and the second tissue region of the first mask image, a first center of the first tissue region, and a second center of the second tissue region;
calculate, using an average value of coordinates of the third tissue region and fourth tissue region of the second mask image, a third center of the third tissue region, and a fourth center of the fourth tissue region;
determine the center line of the first mask image using a connecting line between the first center and the second center and the central axis; and
determine the center line of the second mask image using a connecting line between the third center and the fourth center and the central axis.

26. The medical imaging system as claimed in claim 16, wherein the image generation circuitry is further configured to:
generate a first imaged image of the site of interest in a first phase of the cardiac cycle;
generate a second imaged image of the site of interest at the trigger time; and
subtract the first imaged image from the second imaged image to generate the blood vessel imaged image of the site of interest.

27. A non-transitory computer-readable medium having instructions stored thereon that, when executed by one or more processors of a medical imaging system, cause the medical imaging system to:
scan a site of interest of an examined object to obtain a plurality of frames of initial scanned images of a cross section of the site of interest at different times during a first cycle;
compare each of the plurality of frames of the initial scanned images to one another to identify a plurality of blood vessel locations in the plurality of frames of the initial scanned images;
calculate a trigger time for triggering the scanning of the medical imaging system in a second cycle using signal strengths associated with each of the plurality of blood vessel locations in the plurality of frames of the initial scanned images;
combine the plurality of frames of the initial scanned images to determine a scanning range for operating the medical imaging system to scan the site of interest during the second cycle; and
generate a blood vessel imaged image of the site of interest during the second cycle using the trigger time and the scanning range.
